(19) United States
(12) Patent Application Publication
(10) Pub. No.: US 2018/0256026 A1
(43) Pub. Date: Sep. 13, 2018

(54) INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventor: Osamu Sagano, Inagi-shi (JP)

(21) Appl. No.: 15/909,822

(22) Filed: Mar. 1, 2018

(30) Foreign Application Priority Data
Mar. 9, 2017 (JP) 2017-044605

(51) Int. Cl. A61B 3/12; A61B 3/10; A61B 3/15; G06T 7/20

(52) U.S. Cl. CPC A61B 3/12; A61B 3/102; G06T 2207/30041; G06T 7/20; G06T 2207/10101; A61B 3/152

(57) ABSTRACT

An information processing apparatus includes a scan control unit configured to control a scan with measurement light for generating a three-dimensional motion contrast image of the fundus, and a display control unit configured to display information indicating a progress of the scan on a display unit.

### BACKGROUND OF THE INVENTION

### Field of the Invention

The present invention relates to an information processing apparatus, an information processing method, and a storage medium.

### Description of the Related Art

Various types of ophthalmologic apparatuses employing optical devices have conventionally been used. Optical devices include anterior eye portion cameras, fundus cameras, and Scanning Laser Ophthalmoscopes (SLOs). There is also known an optical tomographic imaging apparatus based on Optical Coherence Tomography (OCT) using multi-wavelength light wave interference. An OCT-based optical tomographic imaging apparatus is capable of obtaining a high-resolution tomographic image of a sample, and is becoming indispensable as an ophthalmologic apparatus for out-patient clinics specialized in retina. In recent years, OCT Angiography (hereinafter referred to as OCTA) has been used as a method for visualizing capillary vessels without using an angiography agent in OCT. As discussed in Japanese Unexamined Patent Application Publication No. 2015-515894, OCTA is a method for visualizing thin blood vessels such as capillary vessels. According to this method, a scan with measurement light is performed at the same position on the retina a plurality of times to detect motions of scattering particles such as red corpuscles.

### SUMMARY OF THE INVENTION

According to an aspect of the present invention, an information processing apparatus includes a scan control unit configured to control a scan with measurement light for generating a three-dimensional motion contrast image of a fundus, and a display control unit configured to display information indicating a progress of the scan on a display unit.

Further features of the present invention will become apparent from the following description of exemplary embodiments with reference to the attached drawings.

### BRIEF DESCRIPTION OF THE DRAWINGS

FIG. 1 illustrates an entire imaging system.

FIG. 2 illustrates a configuration of an optical head and a base unit.

FIG. 3 illustrates a scanning method in Optical Coherence Tomography Angiography (OCTA).

FIG. 4 illustrates a software configuration of an information processing apparatus.

FIGS. 5A and 5B illustrate processing on an EnFace image.

FIG. 6 illustrates an example of a measurement screen.

FIG. 7 is a flowchart illustrating imaging control processing.

FIGS. 8A, 8B, and 8C illustrate examples of progress display.

FIGS. 9A, 9B, and 9C illustrate examples of progress display.

FIG. 10 is a flowchart illustrating imaging control processing according to a second exemplary embodiment.

FIGS. 11A, 11B, and 11C illustrate examples of progress display.

FIGS. 12A and 12B illustrate examples of progress display.

FIGS. 13A, 13B, and 13C illustrate examples of progress display.

FIG. 14 illustrates an example of progress display.

FIG. 15 illustrates an example of progress display.

### DESCRIPTION OF THE EMBODIMENTS

In Optical Coherence Tomography Angiography (OCTA), a volume scan is acquired by scanning the same position a plurality of times, and therefore the imaging time tends to be longer than conventional volume scan. A long imaging time may cause fatigue of the subject eye, and accordingly imaging may not be normally performed because of blink, pupil vignetting, or face movement. To prevent this situation, it is beneficial for an operator to talk to a subject while monitoring the progress of imaging. However, there has been an issue that the operator cannot understand the progress of imaging.

The technique to be discussed has been devised in view of such an issue to allow an operator to understand the progress of imaging.

A first exemplary embodiment of the present invention will be described below with reference to the accompanying drawings.

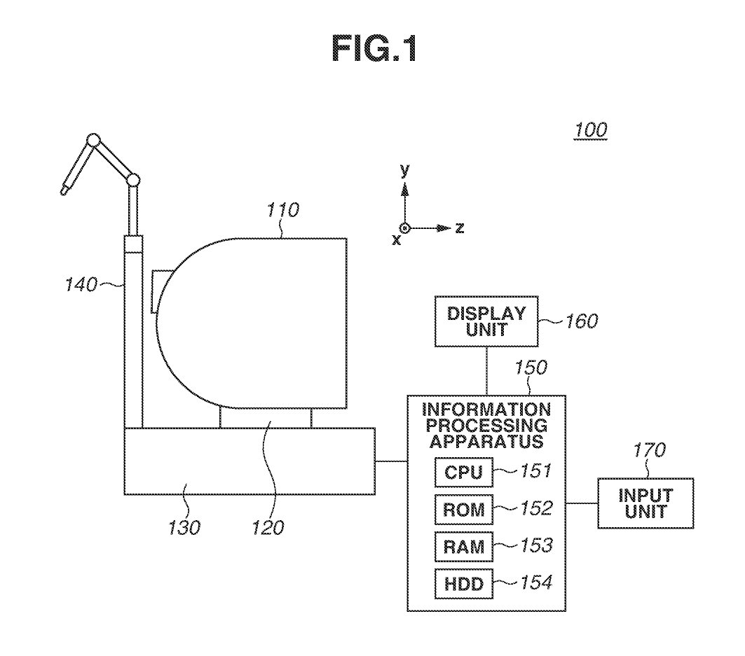

FIG. 1 illustrates an entire imaging system 100 according to the present exemplary embodiment. The imaging system 100 captures an optical interference tomographic image based on Optical Coherence Tomography (OCT) using multi-wavelength light wave interference. An optical head 110 is a measurement optical system for capturing an image of the anterior eye portion and a two-dimensional image and a tomographic image of the fundus. A stage unit 120 is a moving unit for moving the optical head 110 in the xyz directions by using a motor (not illustrated). A base unit 130 includes a spectroscope (described below). A jaw stand 140 fixes the jaw and forehead of the subject. This encourages the fixation of the subject eye.

An information processing apparatus 150 performs tomographic image construction and controls the stage unit 120. A display unit 160 displays various information. An input unit 170 is a keyboard, a mouse, etc. and receives a user operation. In the information processing apparatus 150, a central processing unit (CPU) 151 reads a control program stored in a read only memory (ROM) 152 and performs various processing. A random access memory (RAM) 153 is used as a main memory of the CPU 151 and a temporary storage area such as a work area. A hard disk drive (HDD) 154 stores various data and various programs. Functions and processing of the information processing apparatus 150 (described below) are implemented when the CPU 151 reads a program stored in the ROM 152 or HDD 154 and executes the program. Functions and processing of the information processing apparatus 150 (described below) may be implemented by using a processor other than a CPU. For example, a graphics processing unit (GPU) may be used instead of a CPU.

A hardware configuration of the imaging system 100 is not limited to that in the present exemplary embodiment. For example, the display unit 160 and the input unit 170 may be integrally formed with the information processing apparatus 150, as a touch panel apparatus and so on. In addition, the information processing apparatus 150 may be integrally formed with the base unit 130.

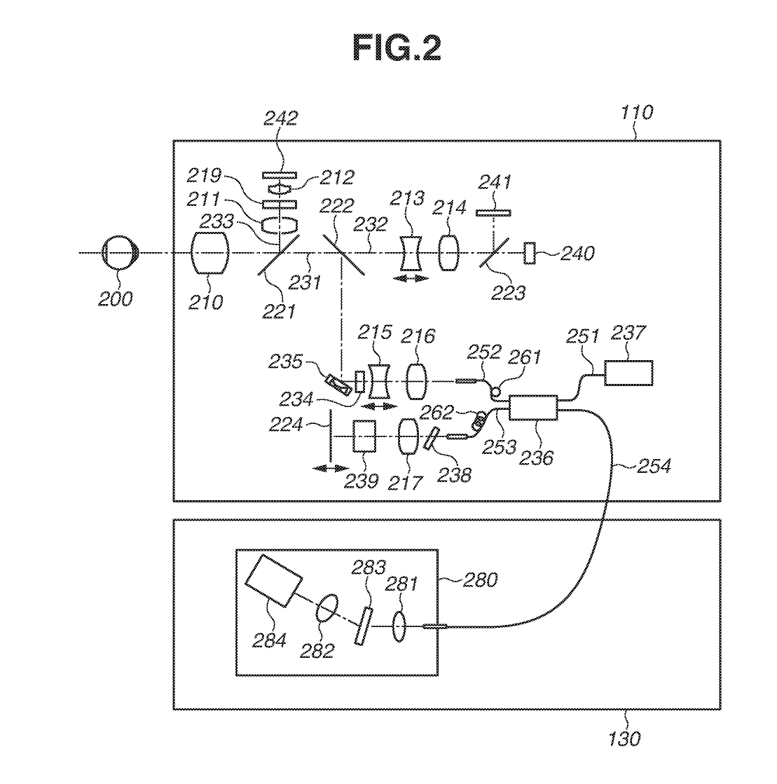

FIG. 2 illustrates a configuration of the measurement optical system and the spectroscope in the optical head 110 and the base unit 130. The configuration of the optical head 110 will be described below. An object lens 211 is disposed to face a subject eye 200. On the optical axis, a first dichroic mirror 221 and a second dichroic mirror 222 branch an optical path into an optical path 231 of the OCT optical system, an optical path 232 for fundus observation and a fixation lamp, and an optical path 233 for anterior eye portion observation, according to a wavelength band.

Likewise, a third dichroic mirror 223 further branches the optical path 232 into an optical path for a charge couple device (CCD) 241 for fundus observation and an optical path for a fixation lamp 240, according to a wavelength band. The optical head 110 includes lenses 213 and 214. The lens 213 is driven by a motor (not illustrated) for focusing for the fixation lamp 240 and fundus observation. The CCD 241 has sensitivity to the wavelength of illumination light for fundus observation (not illustrated), more specifically, around 780 nm. The fixation lamp 240 generates visible light to encourage the fixation of the subject eye. The optical path 233 includes lenses 210 and 211, a slit prism 219, and an infrared CCD 242 for anterior eye portion observation. The CCD 242 has sensitivity to the wavelength of illumination light for anterior eye portion observation (not illustrated), more specifically, around 970 nm.

The optical path 231 configures an OCT optical system as described above to capture a tomographic image of the fundus of the subject eye 200. More specifically, the optical path 231 is used for acquiring an interference signal for forming a tomographic image. A shutter 234 allows the light irradiation to the subject eye only during the imaging time. An XY scanner 235 performs a scan with light on the fundus. The XY scanner 235 illustrated as one mirror scans in 2-axis (XY) directions. The optical head 110 includes lenses 215 and 216. The lens 215 is driven by a motor (not illustrated) to focus, on the fundus of the subject eye 200, the light from a light source 237 emitted from an optical fiber 252 connected to an optical coupler 236. In this focusing, light from the fundus is simultaneously focused in spot form and incident at the end of the optical fiber 252.

A configuration of the optical path from the light source, a reference optical system, and a spectroscope will be described below. The optical head 110 further includes a light source 237, a mirror 224, and a density filter 238 connected to a motor (not illustrated). The density filter 238 rotates to change the amount of transmitted light. The optical head 110 further includes single mode optical fibers 251 to 254 connected to and integrated with the optical coupler 236. The optical head 110 further includes a lens 217 and a spectroscope 280. These components configure a Michelson interference system. Light emitted from the light source 237 passes through the optical fiber 251 and is divided into measurement light on the side of the optical fiber 252 and reference light on the side of the optical fiber 253 via the optical coupler 236.

The measurement light passes through the optical path of the above-described OCT optical system, illuminates the fundus of the subject eye 200 as an observation target, and reaches the optical coupler 236 via the same optical path through reflection and dispersion on the retina. On the other hand, the reference light passes through the optical fiber 253, the density filter 238, the lens 217, and the dispersion compensation glass 239 inserted to adjust the dispersions of the measurement light and reference light, and reaches the mirror 224 to be reflected thereby. Then, the reference light returns along the same optical path and reaches the optical coupler 236.

The measurement light and reference light are coupled by the optical coupler 236 to become interference light. When the light path length of the measurement light becomes almost the same as the light path length of the reference light, interference occurs. The mirror 224 is held to be adjustable in the optical axis direction by a motor and drive mechanism (not illustrated), so that the light path length of the reference light can be adjusted with the light path length of the measurement light which depends on the subject eye 200. The interference light is led to the spectroscope 280 via the optical fiber 254.

The optical head 110 includes a polarization adjustment portion 261 on the measurement light side disposed in the optical fiber 252, and a polarization adjustment portion 262 on the reference light side disposed in the optical fiber 253. The polarization adjustment portions 261 and 262 include some portions of optical fibers being winded in loop form. In the polarization adjustment portions 261 and 262, the loop portions are rotated centering on the fiber longitudinal direction to apply torsion to respective fibers, enabling adjusting polarization conditions of the measurement light and reference light. In this apparatus, it is assumed that the polarization conditions of the measurement light and reference light have been adjusted and fixed in advance.

The spectroscope 280 includes lenses 281 and 282, a diffraction grating 283, and a line sensor 284. After the interference light emitted from the optical fiber 254 becomes parallel light via the lens 281, the parallel light is spectrally dispersed by the diffraction grating 283 and focused on the line sensor 284 by the lens 282.

The periphery of the light source 237 will be described below. The light source 237 is a Super Luminescent Diode (SLD) as a typical low coherent light source. The light source 237 has a central wavelength of 855 nm and a wavelength bandwidth of about 100 nm. The bandwidth is a significant parameter since it influences the resolution (in the optical axis direction) of the tomographic image to be obtained. Although an SLD is selected as the type of the light source 237, Amplified Spontaneous Emission (ASE) is also applicable since it is only necessary to emit low coherent light. Near-infrared light is suitable as the central wavelength, taking the eye measurement into consideration. Further, since the central wavelength influences the horizontal resolution of a tomographic image to be obtained, it is desirable that the central wavelength is an as short wavelength as possible. For both reasons, the central wavelength was set to 855 nm.

Although, in the present exemplary embodiment, a Michelson interferometer was used as an interferometer, a Mach-Zehnder interferometer is also usable. When there is a large difference in light quantity between the measurement light and the reference light, the use of a Mach-Zehnder interferometer is desirable. On the other hand, when there is a small difference in light quantity therebetween, the use of a Michelson interferometer is desirable.

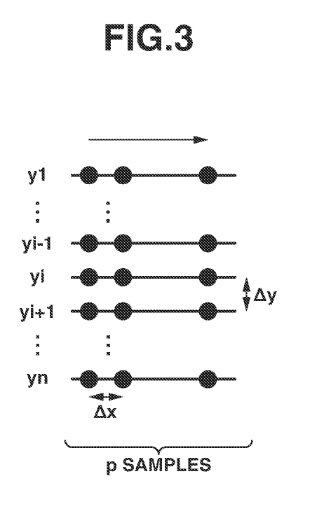

FIG. 3 illustrates a scanning method of OCT Angiography (OCTA). In OCTA, since this method measures time change of an OCT interference signal due to the blood stream, a plurality of measurements is performed at the same location. According to the present exemplary embodiment, the imaging system 100 performs a scan for moving the measurement light to n Y positions while repeating a B scan at the same location m times. As illustrated in FIG. 3, the imaging system 100 repetitively performs a B scan at each of n Y positions (y1 to yn positions) on the fundus plane m times. When m is large, the number of measurements at the same location increases to improve the detection accuracy (S/N) of the blood stream. On the other hand, the scanning time prolongs, and the eye motion (fixation slight motion) during scanning causes an issue of a motion artifact occurrence and an issue of an increase in burden on the subject.

The number of repetitions m may be determined according to the A scan speed and the moving amount of the subject eye 200. The character p indicates the number of times of A scan sampling in a single B scan. More specifically, the plane image size is determined by p×n. When p×n is large and the measurement pitch (Δx, Δy) is the same, it is possible to scan a wide range. In this case, however, the scanning time prolongs, causing the above-described issue of a motion artifact occurrence and issue of an increase in burden on the subject.

Δx indicates the interval (x pitch) between adjacent X positions, and Δy indicates the interval (y pitch) between adjacent Y positions. According to the present exemplary embodiment, the x pitch is set to a half of the beam spot diameter of irradiation light on the fundus, i.e., 10 μm. Even if a pitch is set to be smaller than a half of the beam spot diameter on the fundus, the effect of increasing the definition of an image to be generated is small. Similar to Δx, Δy is also set to 10 μm. Although Δy may be set to be larger than 10 m to reduce the scanning time, it is preferable that Δy does not exceed 20 μm which is the beam spot diameter. As for the x and y pitches, although increasing the beam spot diameter on the fundus degrades the definition, an image of a wide range can be acquired with a small volume of data. The x and y pitches may be freely changed according to clinical demands. Although, in the example illustrated in FIG. 3, horizontal scanning is firstly performed and vertical scanning is secondly performed, the order of scanning may be reversed.

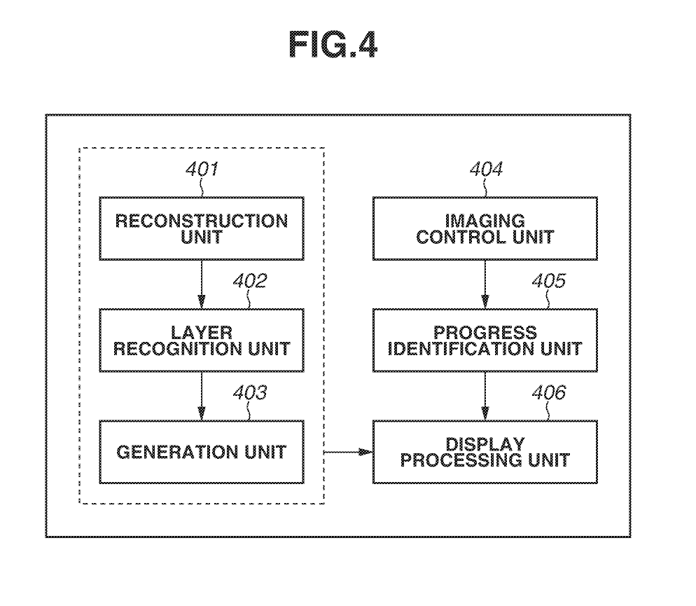

FIG. 4 illustrates a software configuration of the information processing apparatus 150. A reconstruction unit 401 obtains a tomographic image of a predetermined range of an object under measurement based on the interference light generated by the interference between the measurement light returned from the object under measurement and the reference light. More specifically, the reconstruction unit 401 performs wave number conversion and Fast Fourier Transform (FFT) processing on output values from the line sensor 284 to reconstruct a tomographic image (A scan image) in the depth direction at a point on the fundus of the subject eye.

A layer recognition unit 402 extracts retinal layer structures based on a two-dimensional tomographic image, and identifies the shape of each layer boundary. Retina layers to be identified include the retinal nerve fiber layer (RNFL), ganglion cell layer (GCL), inner nuclear layer (INL), outer nuclear layer+inner segment (ONL+IS), outer segment (OS), retinal pigment epithelium (RPE), and Bruch's membrane (BM). A generation unit 403 selects predetermined pixels for each pixel sequence from respective pixel value sequences (in the depth direction) of the tomographic image obtained by the reconstruction unit 401, and generates a two-dimensional image. Examples of two-dimensional images include projection images, EnFace images, and OCTA images. A projection image is generated by integrating predetermined pixels for each pixel sequence from respective pixel value sequences (in the depth direction) of the tomographic image obtained by the reconstruction unit 401.

Each function of the information processing apparatus 150 (described above with reference to FIG. 4) may be implemented through a collaboration of a plurality of CPUs, a ROM, and a RAM or implemented by hardware circuitry.

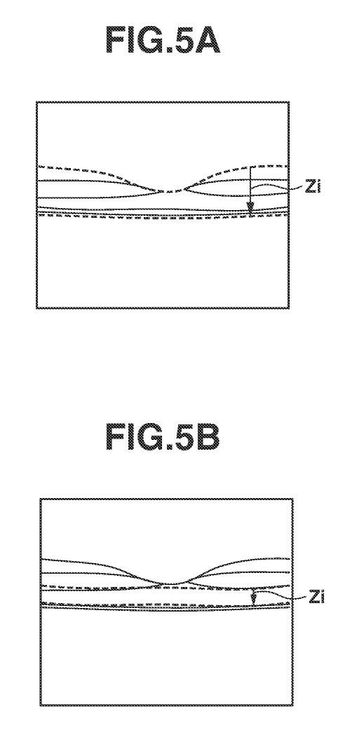

A method for processing an EnFace image will be described below with reference to FIGS. 5A and 5B. The layer recognition unit 402 extracts retinal layer structures based on a two-dimensional tomographic image and identifies the shape of each layer boundary. Although the shape of RPE or presumed RPE (BM) is generally used in an EnFace image in many cases, an arbitrary shape acquired through image analysis, such as cornea shape, is also usable. An identified layer shape is input to the generation unit 403 together with a tomographic image. The generation unit 403 may be configured so that an image acquisition unit generates an EnFace image by using a layer shape and a tomographic image acquired from an external apparatus (not illustrated). In this case, a layer shape and a tomographic image are directly input from the image acquisition unit without interventions of the reconstruction unit 401 and the layer recognition unit 402.

Based on the input boundary layer shape, the generation unit 403 sets a depth range Zi for generating an EnFace image in each A scan image that constitutes a tomographic image. The generation unit 403 sets depth ranges Zi for all of A scan images Ai. The generation unit 403 calculates the average value, maximum value, and median value of tomographic images included in the set depth ranges and then generates an EnFace image by using two-dimensional distributions of these values.

An OCTA image is generated by using de-correlation values between tomographic images obtained by scanning the same portion a plurality of times. Also in OCTA image generation, similar to the method for processing an EnFace image, the layer recognition unit 402 extracts retinal layer structures from a two-dimensional tomographic image and identifies the shape of each layer boundary. Based on the boundary shapes, the generation unit 403 sets a depth range Zi for calculating OCTA in each A scan image that constitutes a tomographic image. The generation unit 403 further calculates a two-dimensional OCTA image by calculating the average value in the depth direction of de-correlation values between tomographic images included in the depth range Zi.

The imaging control unit 404 controls the optical head 110 and the base unit 130. The imaging control unit 404 controls, for example, the scanning with the measurement light. A progress identification unit 405 identifies the scan progress, i.e., the progress of an OCTA image, through control of the imaging control unit 404. A display processing unit 406 controls display on the display unit 160.

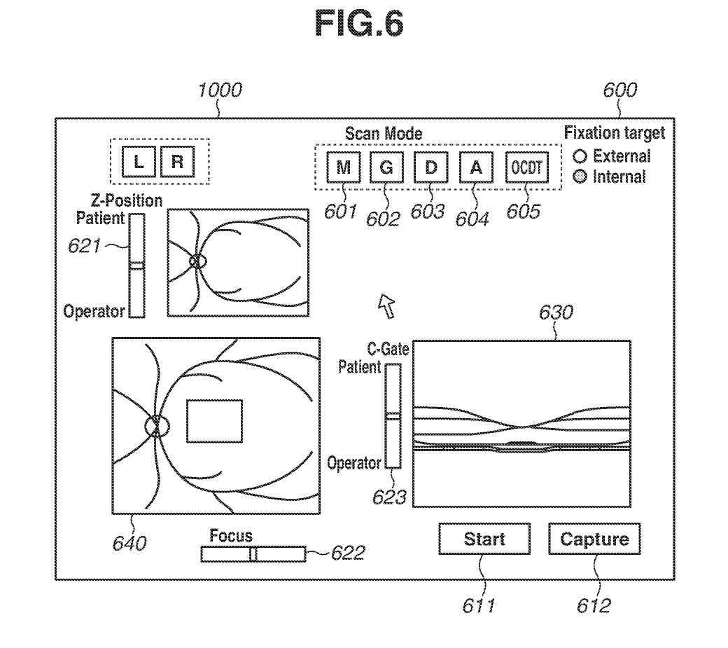

FIG. 6 illustrates an example of an OCTA image measurement screen 600. The measurement screen 600 is displayed on the display unit 160 by the display processing unit 406. A user first selects scan mode buttons 601 to 605: a Macula3D button 601, a Glaucoma3D button 602, a Disc3D button 603, and an Anterior3D button 604, and an OCTA button 605. When the user selects any one of the scan mode buttons 601 to 605, the imaging control unit 404 of the information processing apparatus 150 selects the scan mode corresponding to the selected one of the scan mode buttons 601 to 605. Then, the imaging control unit 404 sets a scanning pattern and a fixation position most suitable for the selected scan mode. Scanning patterns includes a 3D scan, radial scan, cross scan, circle scan, and raster scan.

When the user presses a Start button 611, the imaging control unit 404 automatically performs focal/alignment adjustment. When performing fine focal/alignment adjustment, the imaging control unit 404 moves the position of the optical head 110 in the z direction relative to the subject eye in response to an operation on a slider 621. The imaging control unit 404 also performs focal adjustment in response to an operation on a slider 622 and performs position adjustment on a coherence gate in response to an operation on a slider 623. Then, when the user presses a Capture button 612, the imaging control unit 404 performs control to start imaging. When a result of image capturing is obtained, a tomographic image 630 is displayed as illustrated in FIG. 6. An SLO image 640 is obtained by capturing the imaging range corresponding to an imaging target OCTA image. An SLO image is captured and displayed on the measurement screen 600 before OCTA image capturing.

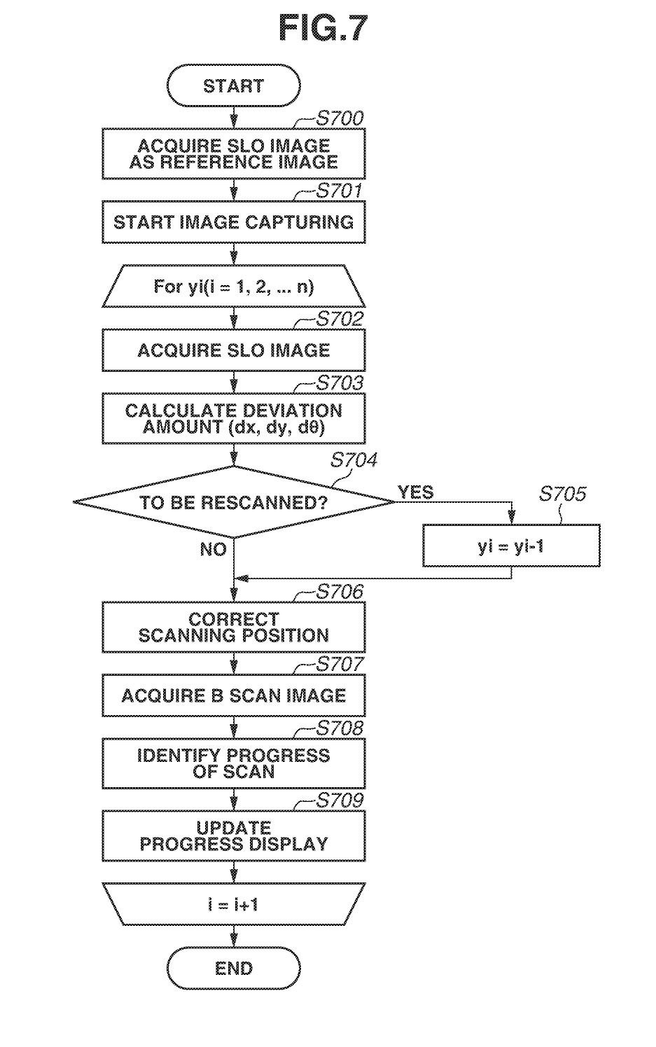

FIG. 7 is a flowchart illustrating imaging control processing by the information processing apparatus 150. The imaging system 100 according to the present exemplary embodiment has a tracking function and a rescan function for capturing an OCTA image of a correct portion even when the fixation of the subject eye moves. The tracking function is a function of changing, when the subject eye moves, the scanning position following the subject eye motion. The rescan function is a function of performing a scan again in a case where a position different from the target position has been scanned due to a tracking delay. An SLO image is used for tracking and rescan. In imaging control processing, the information processing apparatus 150 performs OCTA image capturing while performing tracking and rescan.

In step S700, before OCTA image capturing is performed, the imaging control unit 404 acquires an SLO image as a reference image. An SLO image is an example of an image obtained by capturing the imaging range corresponding to an OCTA image (three-dimensional motion contrast image) obtained in imaging control processing. In step S701, the imaging control unit 404 starts OCTA image capturing. The imaging control unit 404 performs B scan image capturing while sequentially changing the scanning line from y1 to yn. The number of scanning lines and the interval between lines are predetermined. This processing is an example of scan control processing for controlling a scan with the measurement light.

In step S702, during imaging, the imaging control unit 404 acquires an SLO image as an observation image. In step S703, the imaging control unit 404 performs image processing to calculate the amount of positional deviation between the reference image and the SLO image acquired in step S702. In step S704, based on the deviation amount, the imaging control unit 404 determines whether the processing target area (scanning line) is to be rescanned. More specifically, the imaging control unit 404 compares the deviation amount calculated in step S703 with a threshold value as a preset value. The deviation amount equal to or larger than the threshold value means that the B scan position acquired last is not correct. In this case, the imaging control unit 404 changes the scanning line back to the last line to correct the scanning position and then perform a rescan. Therefore, when the deviation amount is equal to or larger than the threshold value, the imaging control unit 404 determines that the processing target area is to be rescanned.

When the imaging control unit 404 determines that the processing target areas is to be rescanned (YES in step S704), the processing proceeds to step S705. On the other hand, when the imaging control unit 404 determines that the processing target area is not to be rescanned (NO in step S704), the processing proceeds to step S706. In step S705, the imaging control unit 404 changes the scanning line back to the last line. In a case where the current scanning line is Y4, for example, in step S705, the imaging control unit 404 changes the scanning line from Y4 to Y3, then the processing proceeds to step S706. Although, in the present exemplary embodiment, the imaging control unit 404 changes the scanning line back to the last line when performing a rescan, the line position to be rescanned is not limited to the last line.

The line position to be rescanned may be determined according to one frame time of an SLO image for determining a rescan and the time interval between B scans in OCTA. For example, in a case where a single frame includes 10 B scans, it is desirable, in rescanning, to change the line position back to the B scan position by 10 lines. In another example, the imaging control unit 404 may set B scans included in a single frame of the corresponding SLO image as rescan targets, instead of the processing target scanning lines.

In step S706, the imaging control unit 404 corrects the scanning position based on the deviation amount calculated in step S703. More specifically, the imaging control unit 404 sets, as a new scanning position, a position offset by the deviation amount calculated in step S703. In step S707, the imaging control unit 404 acquires a B scan image. Each time a scanned image (tomographic image) is acquired, the display processing unit 406 updates the display of the tomographic image 630 in the measurement screen 600 to the newly obtained tomographic image. The imaging control unit 404 repeats the processing in steps S702 to S707 while sequentially changing the scanning line from y1 to yn to complete imaging of all scanning areas.

In repeating the processing, the information processing apparatus 150 according to the present exemplary embodiment performs progress display processing for displaying the progress of processing. More specifically, after completion of the processing in step S707, then in step S708, the progress identification unit 405 of the information processing apparatus 150 identifies the scan progress related to OCTA image capturing. More specifically, the progress identification unit 405 identifies, as a scan progress rate, the ratio of the number of B scans completed till the time of the processing in step S708 to the number of B scans to be performed in OCTA image capturing.

The progress identification unit 405 identifies the remaining time of OCTA image capturing. In a scan, the readout time of the line sensor 284 becomes dominant (rate controlling) at the time of scan processing. According to the present exemplary embodiment, therefore, the progress identification unit 405 calculates the remaining time based on the readout time of the line sensor 284 related to a single A scan. More specifically, in the information processing apparatus 150, “(Readout time of line sensor related to a single A scan)×(Number of repetitions of A scan)×(Number of B scans)” is preset as an OCTA image capturing time. Based on the number of B scans completed till the time of the processing in step S708, the progress identification unit 405 obtains the elapsed time after the start of imaging and calculates the remaining time by subtracting the elapsed time from the imaging time.

In another example, the progress identification unit 405 may obtain the actual elapsed time after the start of imaging. Then, the progress identification unit 405 may recalculate the time used for a single B scan based on the actual elapsed time and the number of B scans completed, change the imaging time based on the recalculated used time, and recalculate the remaining time according to the changed imaging time.

In step S709, the display processing unit 406 performs control to display the progress in an imaging screen and update the display. This processing is an example of display control processing for displaying information about the scan progress on the display unit 160. The CPU 151 changes the scanning line yi by incrementing i by one (up to n) to repeat the processing in steps S702 to S709 for the number of scanning lines. When the processing is completed for up to the scanning line yn, imaging control processing ends.

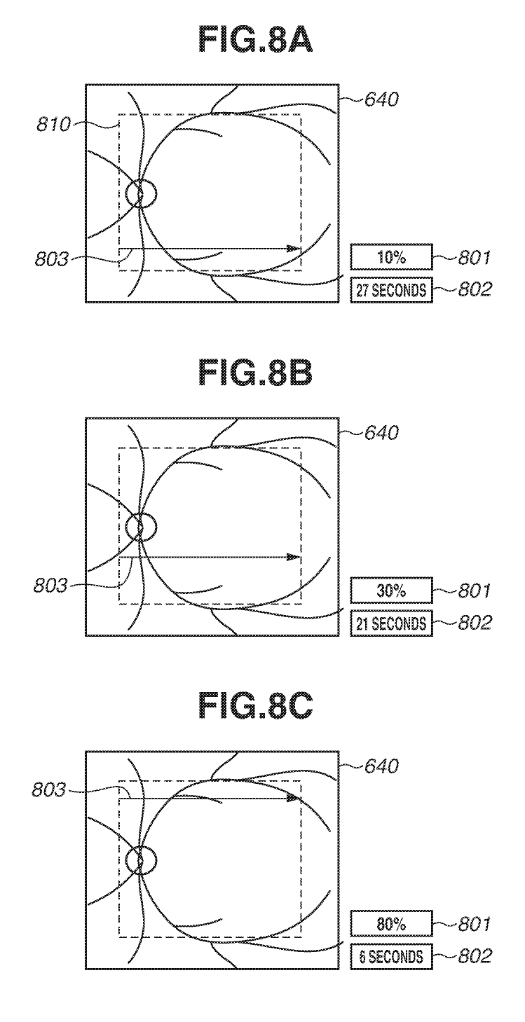

FIGS. 8A to 8C illustrates examples of progress display. According to the present exemplary embodiment, the CPU 151 performs a B scan on an SLO image from left to right of the SLO image and then performs a volume scan while changing the B scan target from the lower side to the upper side of the SLO image. The display processing unit 406 performs control to display, as the scan progress, a progress rate 801 and a remaining time 802 at the bottom right of the SLO image 640 displayed in the measurement screen 600 illustrated in FIG. 6. The display processing unit 406 further performs control to display a scanning range 810 in the SLO image 640, and display, in the scanning range 810, an arrow 803 indicating the processing target scanning line at the time of the processing in step S708.

FIG. 8A illustrates an example of display immediately after starting OCTA image capturing, 10% is displayed as the progress rate 801 and 27 seconds is displayed as the remaining time 802. As imaging progresses, each progress display changes as illustrated in FIGS. 8B and 8C. Referring to FIG. 8B, the progress rate 801 changes to 30% and the remaining time 802 changes to 21 seconds. Referring to FIG. 8C, the progress rate 801 changes to 80% and the remaining time 802 changes to 6 seconds. With the progress of imaging, the display position of the arrow 803 moves upward.

In this way, the imaging system 100 according to the present exemplary embodiment displays the progress of imaging (scanning) at the time of OCTA image capturing. This display allows the operator to know about not only the scan progress but also the timing when scan will end, and therefore assist and talk to the subject. This allows the operator to expect the success rate of imaging to be improved.

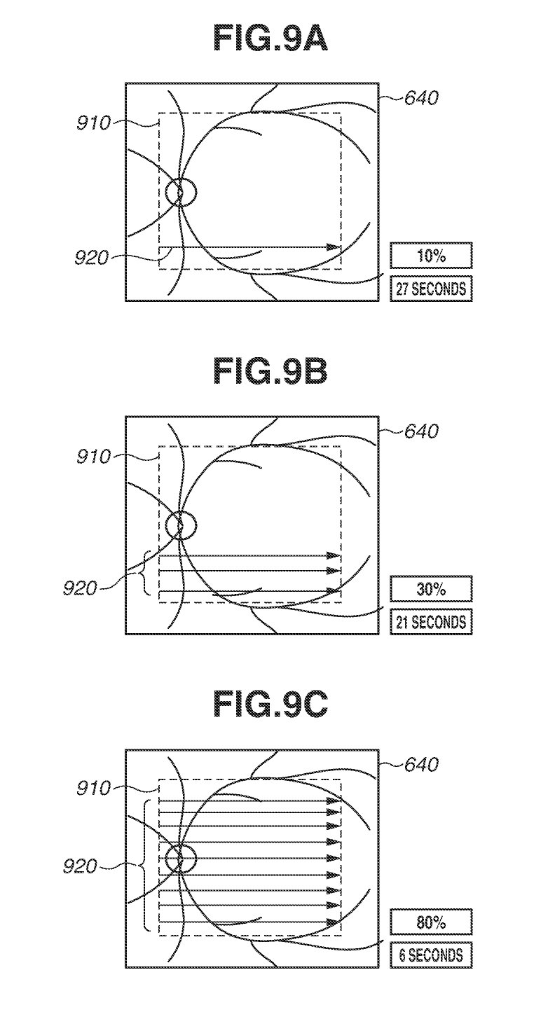

As a first modification of the first exemplary embodiment, the display processing unit 406 may display, on the SLO image 640, an image for distinguishing between an area where scan is completed and an area where scan is not completed. However, the display contents are not limited to the present exemplary embodiment. For example, as illustrated in FIGS. 9A to 9C, the display processing unit 406 may display, with arrows 920, all of scanning lines that have already been scanned in a scanning range 910 in the SLO image 640. Examples of display illustrated in FIGS. 9A to 9C correspond to FIGS. 8A to 8C, respectively. Thus, an observer can intuitively understand the scan progress based not only on numerical values but also on an arrow image indicating the scanning line covered in the scanning range.

As a second modification, information indicating the progress is not limited to the exemplary embodiment. In another example, the total number of lines, the number of scanned lines, the remaining numbers of lines, elapsed processing time, and total processing time in a scan may be displayed in numerical form.

As the second modification, the timing of updating the progress display is not limited to the exemplary embodiment. In another example, the CPU 151 may perform the processing in steps S708 and S709 every other time in repetition of the processing in steps S702 to S707. In another example, the CPU 151 may periodically perform the processing in steps S708 and S709 asynchronously with the repetition of the processing in steps S702 to S707. In another example, the CPU 151 may perform the processing in steps S708 and S709 when a progress display instruction is received in response to a user operation.

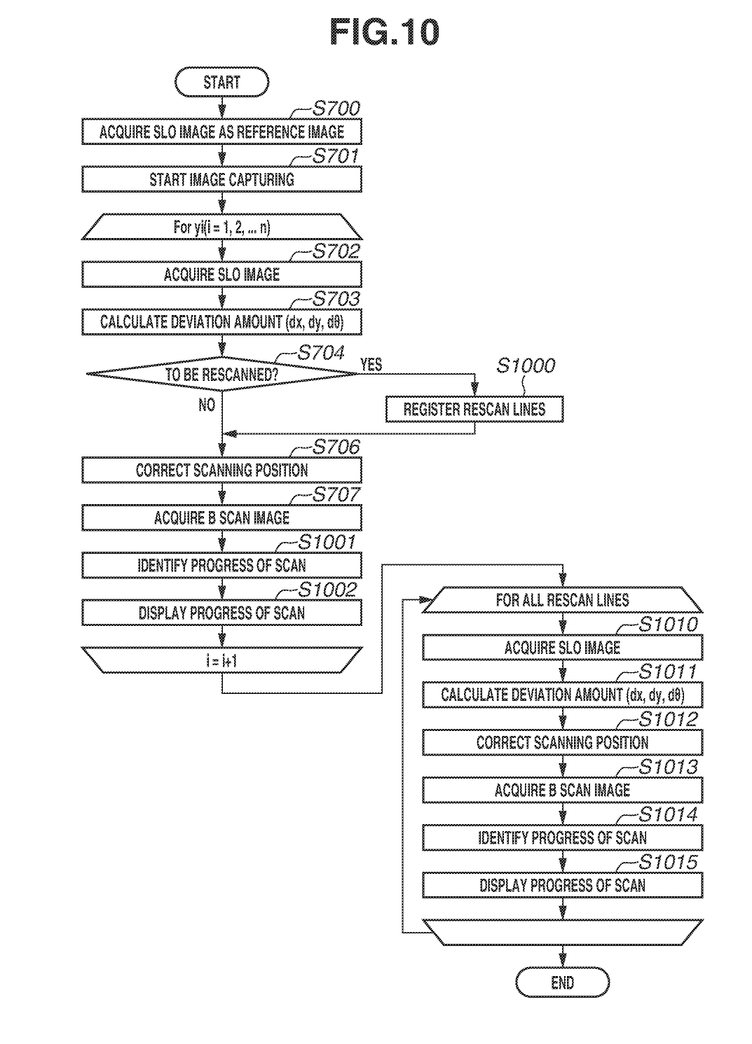

An imaging system 100 according to a second exemplary embodiment will be described below. The imaging system 100 according to the second exemplary embodiment performs a rescan after completion of scanning in all of scanning ranges. The imaging system 100 according to the second exemplary embodiment will be described below centering on differences from the imaging system 100 according to the first exemplary embodiment. FIG. 10 is a flowchart illustrating imaging control processing according to the second exemplary embodiment. Steps in the imaging control processing illustrated in FIG. 10 identical to steps in the imaging control processing according to the first exemplary embodiment described with reference to FIG. 7 are assigned the same reference numerals.

When the CPU 151 determines that the processing target area is to be rescanned (YES in step S704), the processing proceeds to step S1000. In step S1000, instead of performing a rescan, the imaging control unit 404 registers the processing target area as a rescan line (rescan area) to be rescanned, and the processing proceeds to step S706. Information about the rescan line is recorded in a storage unit such as the RAM 153.

After completion of the processing in step S707, the processing proceeds to step S1001. In step S1001, the imaging control unit 404 identifies the scan progress related to OCTA image capturing. According to the present exemplary embodiment, in addition to the scan progress, the imaging control unit 404 calculates processing time used to perform the rescan processing on the rescan line registered in step S1000. In step S1002, the display processing unit 406 performs control to display the scan progress on the imaging screen. According to the present exemplary embodiment, in addition to the scan progress for the scanning area, the display processing unit 406 performs control to display, as a progress, the rescan line and the processing time used to perform the rescan processing.

After completion of repeating the processing in steps S702 to S707, S1001, and S1002, the CPU 151 performs a scan on the rescan line. More specifically, the CPU 151 repetitively performs the processing in steps S1010 to S1015. The processing in steps S1010 to S1013, targeting each of the rescan lines registered in step S1000, is repeated until completion of processing on all of the processing target rescan lines. For convenience of descriptions, hereinafter, the processing in steps S702 to S707, S1001, and S1002 is referred to as scan processing, and the processing in steps S1010 to S1015 is referred to as rescan processing.

The imaging control unit 404 selects one of the rescan lines registered in step S1000 and performs processing in step S1010 and subsequent steps. The processing in steps S1010 to S1013 is scan processing to be performed on the rescan lines, and basic processing is similar to the processing in steps S702 to S707. More specifically, the imaging control unit 404 acquires an SLO image in step S1010, calculates the deviation amount in step S1011, and corrects the scanning position in step S1012. In step S1013, the imaging control unit 404 acquires a B scan image at a new scanning position offset by the deviation amount.

In step S1014, the imaging control unit 404 calculates the processing time in the rescan processing as the scan progress. When the rescan processing has already been started, the remaining processing time is calculated as the processing time. In step S1015, the display processing unit 406 displays the progress and updates the progress display according to the progress identified in step S1014.

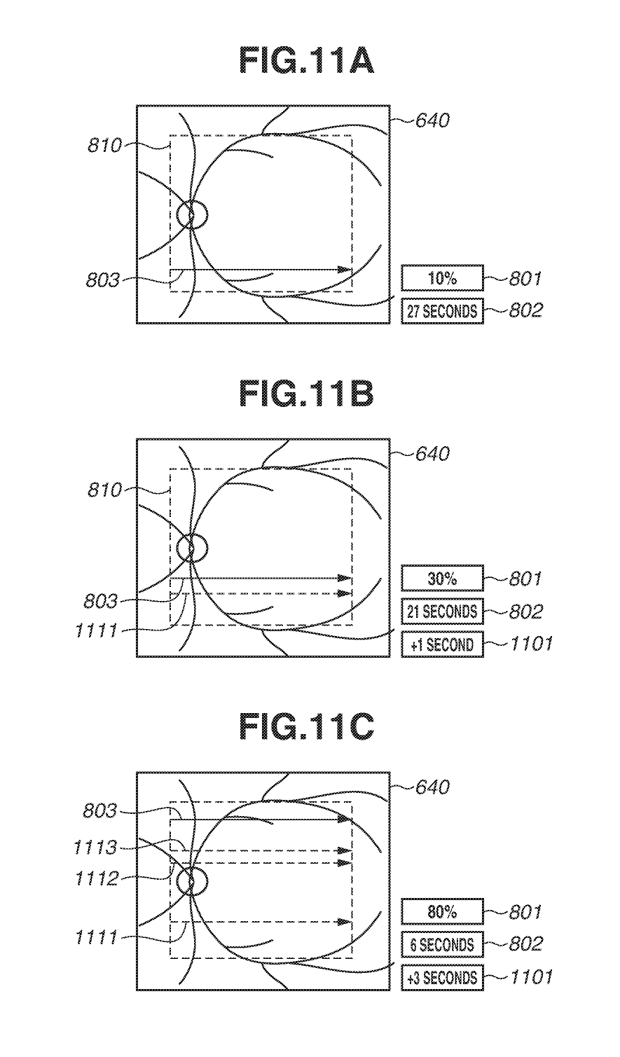

FIGS. 11A to 11C illustrate examples of progress display according to the second exemplary embodiment. FIG. 11A illustrates an example of display immediately after starting OCTA image capturing. At the time of this processing, rescan lines have not yet been registered. In this case, similar to the example of display illustrated in FIG. 8A, the progress rate 801 and the remaining time 802 are displayed. In addition, the arrow 803 indicating the processing target scanning line is displayed on the SLO image 640.

FIG. 11B illustrates an example of display when rescan lines are registered after imaging progresses from the state illustrated in FIG. 11A. In addition to the progress rate 801 and the remaining time 802, “+1 second” is displayed as a rescan processing time 1101. On the SLO image 640, the dotted line arrow 1111 indicating the rescan line is displayed. FIG. 11C illustrates an example of display when the number of rescan lines is increased to three after imaging progresses from the state illustrated in FIG. 11B. In this case, the processing time 1101 increases to “+3 seconds”, and three dotted line arrows 1111 to 1113 are displayed. Other configurations and processing of the imaging system 100 according to the second exemplary embodiment are similar to those of the imaging system 100 according to the first exemplary embodiment.

In this way, at the time of OCTA image capturing, the imaging system 100 according to the second exemplary embodiment displays, as the progress of imaging (scanning), the progress of the scan processing and the progress of the rescan processing at the time of processing. Therefore, together with the progress of the scan processing, the operator can understand the processing time related the rescan processing subsequently performed.

As a modification of the second exemplary embodiment, the imaging control unit 404 may perform further rescan on the processing target scanning line when the deviation amount calculated in step S1011 in the rescan processing is equal to or larger than the threshold value.

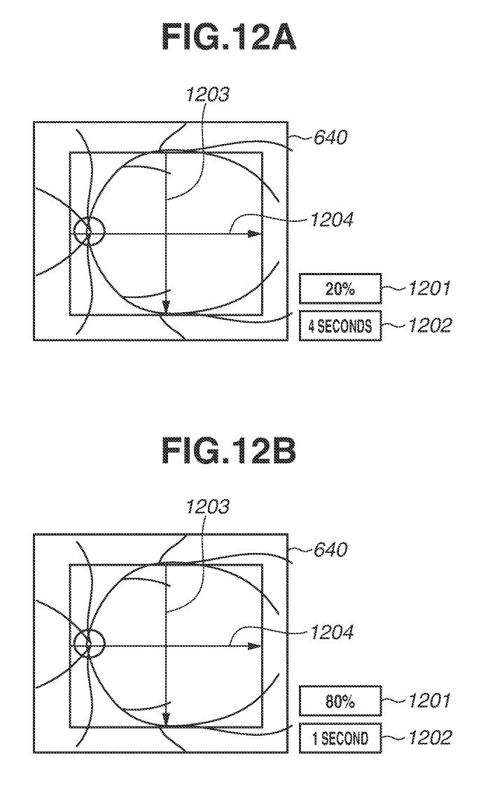

Modifications according to the first and the second exemplary embodiments will be described below. Although, in the above-described exemplary embodiments, tracking and rescan are performed while sequentially changing the scan line one by one for the general volume scan, tracking and rescan are not limited thereto. In another example, similar to cross scan, the above-described exemplary embodiments are also applicable to a case where sampling is performed on the same position a plurality of times. FIGS. 12A and 12B illustrate examples of progress display in a case of cross scan. The examples display arrows 1203 and 1204 indicating the processing target scanning lines in addition to a progress rate 1201 and a remaining time 1202.

Although, in the above-described examples, the scan progress is displayed on an SLO image, the scan progress display is not limited thereto. In another example, instead of an SLO image, the display processing unit 406 may display the scan progress on an anterior eye portion image acquired for anterior eye portion alignment.

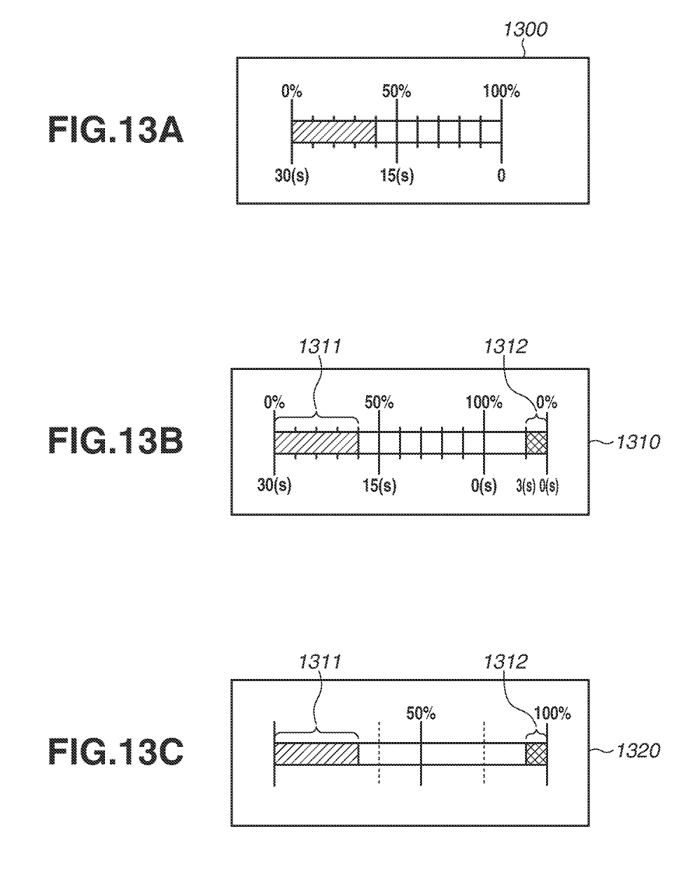

Modifications of progress display will be described below. FIGS. 13A to 13C illustrate a first modification of progress display. In the measurement screen 600, for example, the display processing unit 406 may display a bar graph indicating the progress at a position such as an area under the tomographic image 630. FIG. 13A illustrates a bar graph 1300 according to the first exemplary embodiment. The bar graph 1300 indicates the progress rate and remaining time of the scan processing. Performing progress display in this way allows the operator to understand the scan progress and timely assist and talk to the subject, thus improving the success rate of imaging.

FIG. 13B illustrates a bar graph 1310 according to the second exemplary embodiment. Referring to the bar graph 1310, a portion 1311 extending from the left end indicates the progress of the scan processing. On the right-hand side on the same axis as the progress of the scan processing, a portion 1312 extending from the right end indicates the progress of the rescan processing. In the bar graph related to the rescan processing, the increase direction of the progress rate and remaining time is opposite to the increase direction of the graph of the scan processing.

In another example, as illustrated in FIG. 13C, the bar graph may display the progress rate and remaining time of the scan processing and rescan processing, assuming that the total processing time and the number of scan lines for the scan processing and rescan processing are 100%. Also referring to FIG. 13C, a bar graph 1320 displays scan processing 1311 and rescan processing 1312.

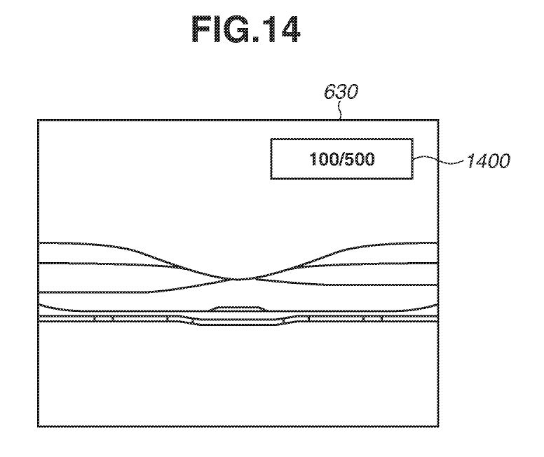

As a second modification of progress display, the display processing unit 406 may display the progress on the tomographic image 630. FIG. 14 illustrates the second modification. In the example illustrated in FIG. 14, the total number of scan lines (500 lines) and the number of scanned lines (100 lines) in the scan processing are displayed as a progress 1400. The operator determines whether the imaging range of the tomographic image is suitable as a barometer in determining a success or failure of imaging. For this determination, the tomographic image 630 to be updated is confirmed. On the other hand, progress display on a tomographic image allows the observer to confirm the progress almost without moving the line of sight.

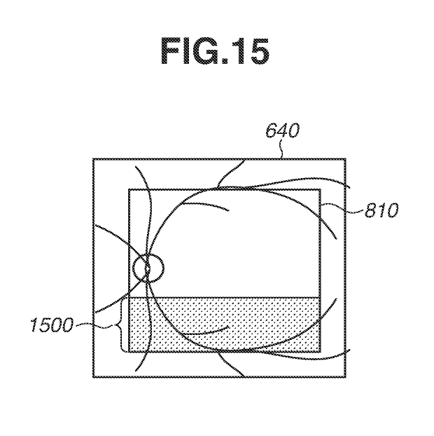

As a third modification of progress display, as illustrated in FIG. 15, the information processing apparatus 150 may generate a projection image from a B scan image (tomographic image) that has been scanned, superimpose and display the projection image on the corresponding area of the SLO image 640. A projection image refers to a two-dimensional projection image in which a tomographic image is projected in the depth direction. An SLO image is an example of a captured image corresponding to a three-dimensional motion contrast image. A projection image is an example of an image which indicates the progress. This processing is an example of display control processing for performing control to display a two-dimensional projection image superimposed on a captured image.

In the example illustrated in FIG. 15, a projection image (OCT image) 1500 is superimposed and displayed in the scanning range 810. As the scan processing progresses, the ratio of the projection image 1500 increases. The above-described superimposed display of the projection image 1500 allows the operator to check whether imaging up to the display time is successful. Although, in the present exemplary embodiment, the display processing unit 406 captures a projection image, the image to be displayed is not limited to a projection image. In another example, the display processing unit 406 may superimpose and display a two-dimensional projection image such as an EnFace and an OCTA image. The display processing unit 406 may display a thick line at the boundary position between the drawing area of the projection image 1500 and the non-drawing area to emphasize the boundary position.

In another example, the imaging system 100 may display the elapsed time, the remaining time, the ratio of completed scans, and the ratio of the remaining scan related to OCTA image capturing (scanning) in the form of numerical values, a bar graph, and a pie chart. In still another example, the imaging system 100 may perform control to change the color and brightness of the arrow indicating the current scanning position according to the scan progress.

While various exemplary embodiments of the present invention have been described, the present invention is not limited thereto but can be modified in diverse ways without departing from the spirit and scope thereof. Portions of the above-described exemplary embodiments may be combined.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-044605, filed Mar. 9, 2017, which is hereby incorporated by reference herein in its entirety.

What is claimed is:

1. An information processing apparatus comprising:
a scan control unit configured to control a scan with measurement light for generating a three-dimensional motion contrast image of a fundus; and
a display control unit configured to display information indicating a progress of the scan on a display unit.

2. The information processing apparatus according to claim 1, wherein the display control unit performs control to superimpose and display an image indicating the progress on a captured image of an imaging range corresponding to the three-dimensional motion contrast image.

3. The information processing apparatus according to claim 2, wherein the display control unit performs control to superimpose and display an image for distinguishing between an area where the scan is completed and an area where the scan is not completed on the captured image.

4. The information processing apparatus according to claim 3, further comprising a generation unit configured to generate a two-dimensional projection image based on a tomographic image of the area where the scan is completed, wherein the display control unit performs control to superimpose and display the two-dimensional projection image generated by the generation unit on a corresponding area of the captured image.

5. The information processing apparatus according to claim 1, wherein the display control unit performs control to display a progress rate as information indicating the progress.

6. The information processing apparatus according to claim 1, wherein the display control unit performs control to display at least either one of an elapsed time and a remaining time related to the scan as information indicating the progress.

7. The information processing apparatus according to claim 1, wherein the display control unit performs control to display a bar graph indicating the progress.

8. The information processing apparatus according to claim 1, wherein the display control unit performs control to display information indicating the progress on a tomographic image obtained as a result of the scan.

9. The information processing apparatus according to claim 1, wherein, in a case where a scanning line where a scan is performed is registered as a line subjected to a rescan, the display control unit performs control to display information indicating a progress related to the rescan.

10. The information processing apparatus according to claim 9, wherein the display control unit performs control to superimpose and display the information indicating the progress related to the rescan on a captured image of an imaging range corresponding to the three-dimensional motion contrast image.

11. The information processing apparatus according to claim 10, wherein the display control unit performs control to superimpose and display an image indicating a scanning line subjected to the rescan on the captured image.

12. The information processing apparatus according to claim 9, wherein the display control unit performs control to display, on a same axis, a bar graph indicating a progress related to the scan and a progress related to the rescan.

13. An information processing method to be performed by an information processing apparatus, the method comprising:
controlling a scan with measurement light for generating a three-dimensional motion contrast image of a fundus; and
displaying information indicating a progress of the scan on a display unit.

14. A non-transitory storage medium storing a program for causing a computer to function as:
a scan control unit configured to control a scan with measurement light for generating a three-dimensional motion contrast image of a fundus; and
a display control unit configured to display information indicating a progress of the scan on a display unit.
